(19) United States
(12) Patent Application Publication
(10) Pub. No.: US 2026/0144532 A1
(43) Pub. Date: May 28, 2026

(54) RELEASE MECHANISM FOR OVER THE SCOPE CLIP

(71) Applicant: BOSTON SCIENTIFIC SCIMED, INC., Maple Grove, MN (US)

(72) Inventors: Ryan Davis GILKEY, Austin, TX (US); Alexander COHEN, Austin, TX (US)

(21) Appl. No.: 19/357,885

(22) Filed: Oct. 14, 2025

Related U.S. Application Data
(60) Provisional application No. 63/726,115, filed on Nov. 27, 2024.

(51) Int. Cl. A61B 17/00; A61B 1/00; A61B 17/04

(52) U.S. Cl. CPC A61B 17/0057; A61B 1/00128; A61B 1/0014; A61B 17/00234; A61B 2017/00296; A61B 2017/00367; A61B 2017/00477; A61B 2017/00584; A61B 2017/00623; A61B 2017/00867; A61B 2017/0488

(57) ABSTRACT

A clipping system includes an adapter, a clip, a coupling, and a release member. The adapter includes a distal portion over which the clip is mounted. Jaws of the clip move between being separated to receive tissue and drawn to grip the tissue. The coupling includes a channel and a recess formed in a side of the coupling. The recess receives a side portion of the clip therewithin and accessible by a window in the side. The member extends partially through the channel and includes a retaining portion positioned within the recess between the window and the side portion. The member moves to a release position to withdraw from the recess to permit the side portion to exit the recess through the window to decouple the clip from the system.

### PRIORITY CLAIM

The present disclosure claims priority to U.S. Provisional Patent Application Serial No. 63/726,115 filed November 27, 2024; the disclosure of which is incorporated herewith by reference.

### FIELD

The present disclosure relates to endoscopic devices and, in particular, relates to endoscopic clipping devices for treating tissue, for example, within the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks). Currently, tissue may be treated via endoscopic closure devices including through-the scope clips or over-the-scope clips (OTSC). Over-the-scope clips may be particularly useful for achieving closure of larger tissue defects. These endoscopic closure devices can save costs for the hospital and may provide benefits for the patient.

Over-the-scope clips can be formed so that the clip is biased toward a deployment configuration in which the jaws of the clip are closed to grip target tissue. These clips are generally navigated to a target region in the body in an insertion configuration in which the jaws of the clip are open, e.g., around an adapter fit around the distal end of a flexible endoscope. The clip may then be moved off the distal end of the adapter so that the natural bias of the clip causes the jaws to close, e.g., around target tissue. Many over-the-scope clips cannot be reopened after an initial deployment, while others can be closed and reopened multiple times so that the clip can be repositioned after an initial deployment. Many repositionable over-the-scope clips in current use require the breaking, failure or separation of a frangible member to decouple the clip from the adapter and fully deploy the clip. While the frangible member remains intact, the clip remains coupled to the adapter so that the clip can be reopened and repositioned, if desired by the operating physician. The frangible member of these existing systems is configured to possess properties such that the member can withstand the internal forces imparted by opening and closing the repositionable clip. Accordingly, in these existing systems, the force applied by the mechanism for separating the clip from the adapter, e.g., breaking the frangible link, may be relatively high.

### SUMMARY

The present disclosure relates to a clipping system for treating tissue. The system includes an adapter including a proximal portion configured to be mounted over a distal end of an insertion device and a distal portion extending distally from the proximal portion; a clip configured to be mounted over the distal portion of the adapter in an insertion configuration, the clip including first and second jaws connected to one another such that the first and second jaws are movable between the insertion configuration, in which the first and second jaws extend about opposing portions of the distal portion of the adapter and are separated from one another to receive tissue therebetween, and an initial deployment configuration, in which the clip is moved distally off of the adapter so that the first and second jaws are drawn toward one another to grip the tissue therebetween, the first and second jaws being biased toward the initial deployment configuration; a distal coupling including a channel extending therethrough and a recess formed in a side of the distal coupling, the recess sized and shaped to receive a side portion of the clip therewithin, the recess accessible by a window in the side of the distal coupling; and a release member sized and shaped to extend at least partially through the channel of the distal coupling, the release member including a retaining portion that, in a retaining position, is positioned within the recess between the window and the side portion of the clip, the retaining portion configured to retain the side portion of the clip within the recess by blocking the side portion of the clip from exiting the recess through the window, the release member movable to a release position in which the retaining portion is withdrawn from the recess to permit the side portion of the clip to exit the recess through the window to decouple the clip from the clipping system.

In an embodiment, the clip includes an opening through which, in the retaining position, the retaining portion of the release member extends, wherein, in the initial deployment configuration, a distal portion of the distal coupling is distal to a first surface of the clip, a proximal portion of the distal coupling is proximal to a second surface of the clip, and the side portion of the clip is received within the recess of the distal coupling.

In an embodiment, the system further includes an extending member fixed at a distal end thereof to a proximal end of the distal coupling. Decoupling the clip from the clipping system comprises withdrawing the extending members proximally so that the distal coupling is withdrawn proximally relative to the clip while the clip remains closed around target tissue.

In an embodiment, the extending members and the release members are movable together proximally or distally by a single actuator when the clip is in the insertion configuration, the initial deployment configuration, and a review configuration, the release members are actuated independently from the extending members to transition to the release position.

In an embodiment, the distal coupling comprises an angled surface on a distal side of the recess that urges the distal coupling to rotate against the side portion of the clip as the distal coupling is withdrawn proximally relative to the clip, the rotation permitting further proximal motion of the distal coupling until the side portion passes outside the recess and the clip is released from the distal coupling.

In an embodiment, a distal portion of the release member including the retaining portion is unanchored, wherein lateral forces against the release member by the side portion of each of the first and second jaws are insufficient to overcome frictional forces between the release member, the side portion of each of the first and second jaws and the distal coupling, wherein the release member includes a lateral stiffness sufficient to withstand torsional forces imposed by the side portion of the clip as the side portion of the clip rotates or translates during transitions between the insertion configuration and the initial deployment configuration.

In an embodiment, the distal portion of the release member includes a hook portion sized and shaped to wrap around the side portion of the clip, the hook portion including a first part on a first side of the side portion, a second part curving around a second side of the side portion, and a third part comprising the retaining portion on the second side of the side portion.

In an embodiment, the first side of the side portion of the clip includes a first groove for guiding the first part of the hook portion of the release member and a third side of the side portion of the clip includes a second groove for guiding the third part of the hook portion of the release member.

In an embodiment, moving the release member to the release position comprises advancing the release member distally so that the third part of the release member is withdrawn from the recess.

In an embodiment, the distal coupling includes a cap portion including a slot, wherein advancing the release member distally comprises advancing the hook portion distally into the slot.

In an embodiment, moving the release member to the release position comprises retracting the release member proximally so that the distal portion of the release member is retracted proximal to the recess.

In an embodiment, the release member is formed of Nitinol wire.

In an embodiment, the release member is formed of suture, wire, or hypotube.

In an embodiment, the release member includes the retaining portion formed of a first material and a proximal portion formed of a second material.

In an embodiment, the clip can be moved into a review configuration in which the clip is moved distally relative to the adapter.

In addition, present disclosure relates to a method for treating tissue including inserting to a target area in a body lumen, a clip mounted over a distal portion of an adapter, the adapter including a proximal portion configured to be mounted over a distal end of an insertion device and the distal portion extending distally from the proximal portion, the clip being maintained in an insertion configuration in which first and second jaws extend about opposing portions of the distal portion of the adapter and are separated from one another to receive tissue therebetween, the clip being coupled to the adapter by a distal coupling including a channel extending therethrough and a recess formed in a side of the distal coupling, the recess sized and shaped to receive a side portion of the clip therewithin, the recess accessible by a window in the side of the distal coupling, and a release member sized and shaped to extend at least partially through the channel of the distal coupling, the release member including a retaining portion that, in a retaining position, is positioned within the recess between the window and the side portion of the clip, the retaining portion configured to retain the side portion of the clip within the recess by blocking the side portion of the clip from exiting the recess through the window; drawing the tissue into a channel of the adapter and between the first and second jaws of the clip; moving the clip distally off of the adapter to permit the clip to move from the insertion configuration to an initial deployment configuration, the first and second jaws of the clip being biased toward the initial deployment configuration in which the first and second jaws extend toward one another to grip the tissue drawn into the adapter; moving the release member to a release position in which the retaining portion is withdrawn from the recess to permit the side portion of the clip to exit the recess through the window; and drawing the distal coupling proximally to decouple the clip from a clipping system.

In an embodiment, the clip includes an opening through which, in the retaining position, the retaining portion of the release member extends, wherein, in the initial deployment configuration, a distal portion of the distal coupling is distal to a first surface of the clip, a proximal portion of the distal coupling is proximal to a second surface of the clip, and the side portion of the clip is received within the recess of the distal coupling.

In an embodiment, a distal portion of the release member including the retaining portion is unanchored, wherein lateral forces against the release member by the side portion of each of the first and second jaws are insufficient to overcome frictional forces between the release member, the side portion of each of the first and second jaws and the distal coupling, wherein the release member includes a lateral stiffness sufficient to withstand torsional forces imposed by the side portion of the clip as the side portion of the clip rotates or translates during transitions between the insertion configuration and the initial deployment configuration.

In an embodiment, the distal portion of the release member includes a hook portion sized and shaped to wrap around the side portion of the clip, the hook portion including a first part on a first side of the side portion, a second part curving around a second side of the side portion, and a third part comprising the retaining portion on the second side of the side portion, wherein moving the release member to the release position comprises advancing the release member distally so that the third part of the release member is withdrawn from the recess.

In an embodiment, moving the release member to the release position comprises retracting the release member proximally so that the distal portion of the release member is retracted proximal to the recess.

### BRIEF DESCRIPTION

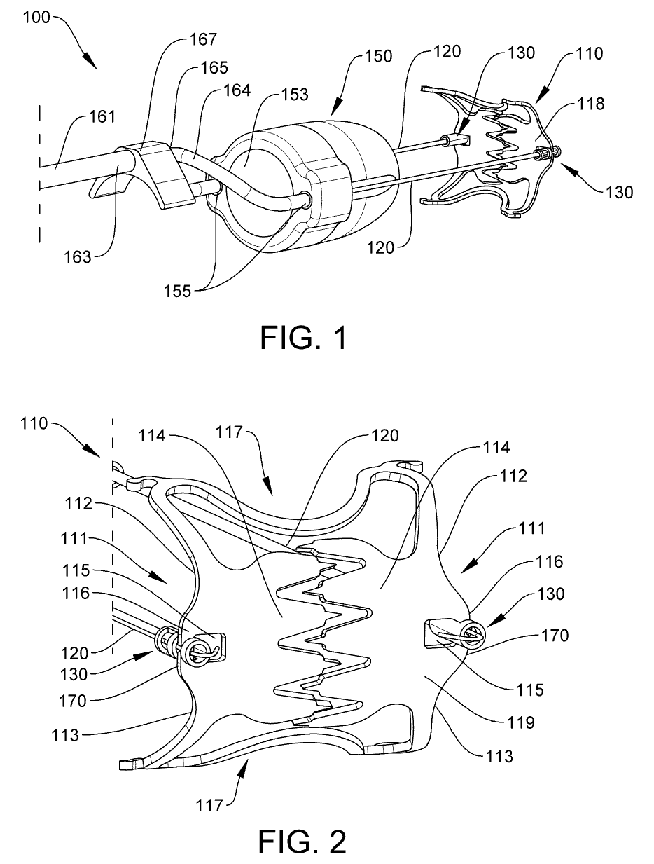

FIG. 1 shows a clipping system according to various exemplary embodiments.

FIG. 2 shows a clip of the clipping system of FIG. 1.

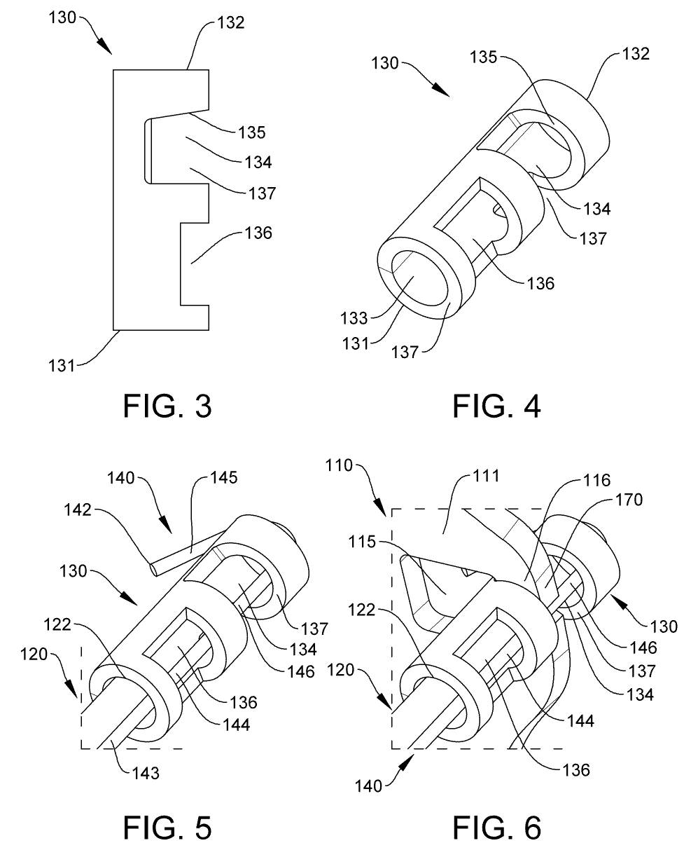

FIG. 3 shows a side view of a distal coupling of the clipping system of FIG. 1.

FIG. 4 shows a perspective view of the distal coupling of FIG. 3.

FIG. 5 shows a perspective view of the distal coupling of FIG. 3 with a release member extending through the distal coupling.

FIG. 6 shows a perspective view of the distal coupling of FIG. 3 with a side portion of a jaw of the clip received in a recess of the distal coupling and the release member retaining the side portion of the jaw within the recess.

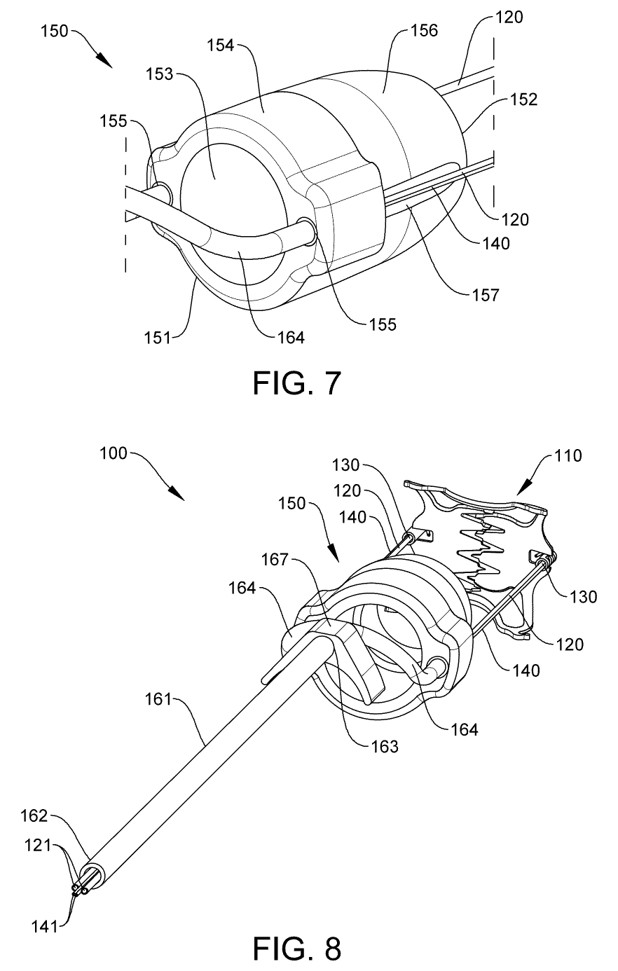

FIG. 7 shows an adapter of the clipping system of FIG. 1.

FIG. 8 shows a proximal end of the clipping system of FIG. 1.

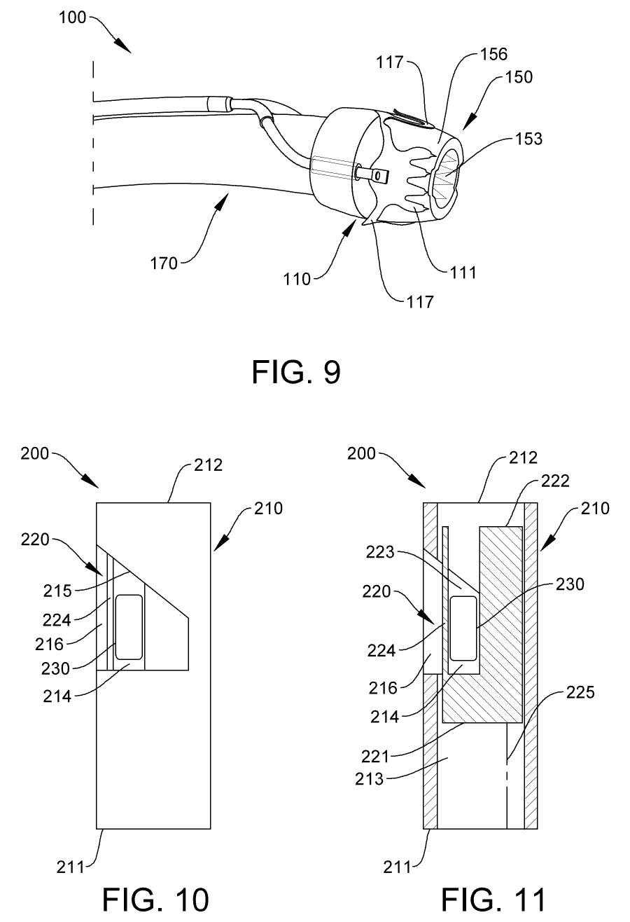

FIG. 9 shows the clip of the clipping system of FIG. 1 in an insertion configuration in which the clip is mounted over a distal portion of the adapter.

FIG. 10 shows a clipping system including a coupling mechanism for a clip comprising nested tubes according to various exemplary embodiments.

FIG. 11 shows a section view of the coupling mechanism of the clipping system of FIG. 10.

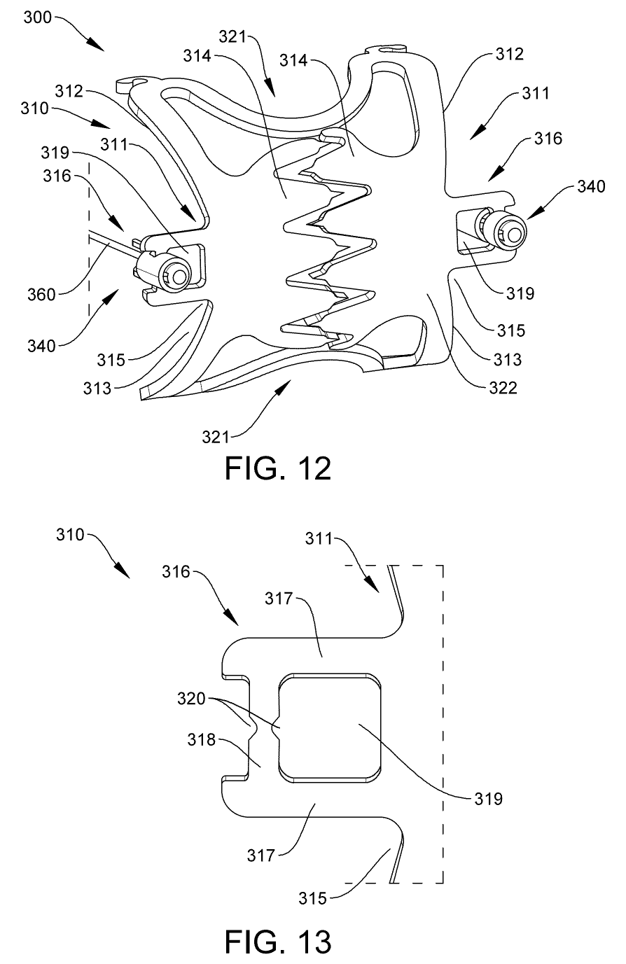

FIG. 12 shows a clipping system according to another exemplary embodiment.

FIG. 13 shows a side portion of a clip of the clipping system of FIG. 12.

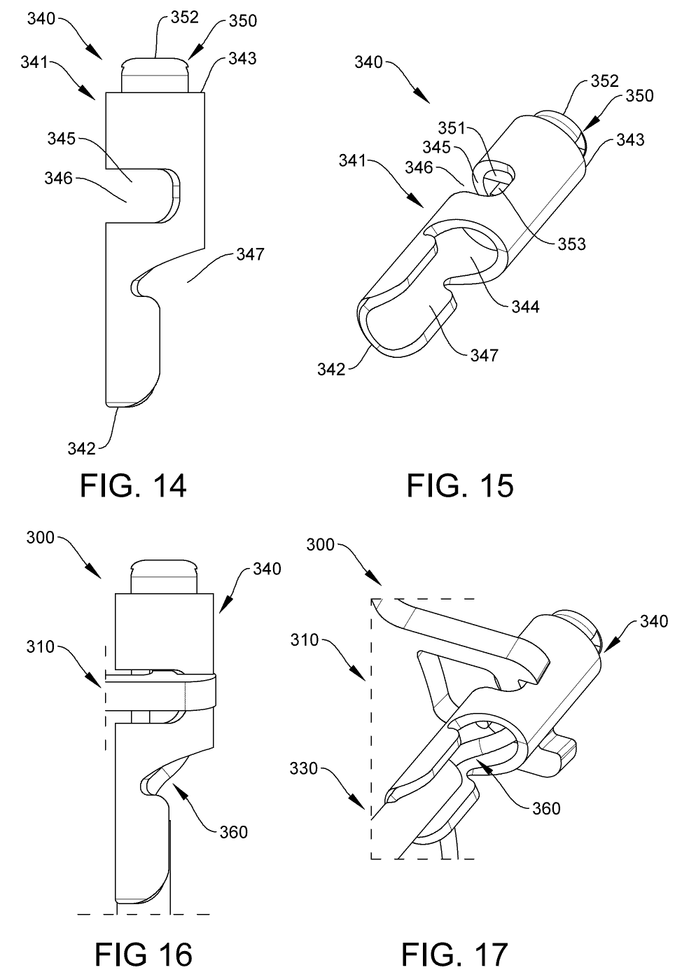

FIG. 14 shows a side view of a distal coupling of the clipping system of FIG. 12.

FIG. 15 shows a perspective view of the distal coupling of FIG. 14.

FIG. 16 shows a side view of a coupling mechanism including the clip, the distal coupling and a release member of the clipping system of FIG. 12.

FIG. 17 shows a perspective view of the coupling mechanism of FIG. 16.

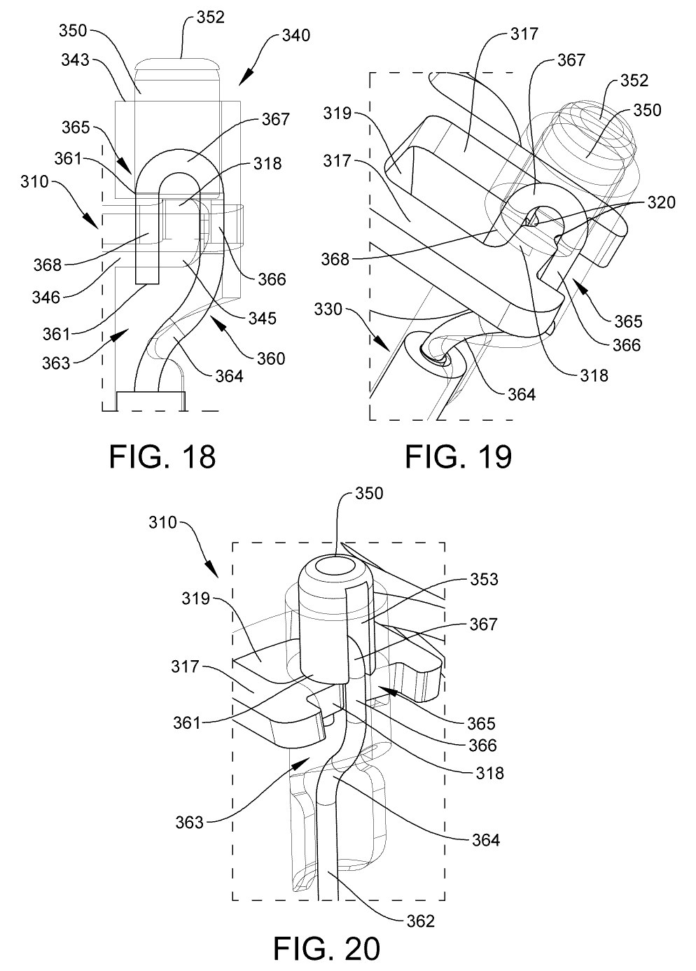

FIG. 18 shows a side view of the coupling mechanism of FIG. 16 with partial transparency.

FIG. 19 shows a first perspective view of the coupling mechanism of FIG. 16 with partial transparency.

FIG. 20 shows a second perspective view of the coupling mechanism of FIG. 16 with partial transparency.

### DETAILED DESCRIPTION

The present disclosure may be further understood with reference to the following description and the appended drawings, wherein like elements are referred to with the same reference numerals. The present disclosure relates to a clipping system, in particular, an over-the-scope endoscopic clipping system, in which an initial placement of a clip may be viewed and, if desired, the clip may be reopened and repositioned prior to a final deployment of the clip. In various exemplary embodiments, the clipping system includes a mechanism for releasing the clip in the final deployment, e.g., decoupling the clip from the clipping system, comprising a release member configured to withstand internal forces imparted thereupon, e.g., torsional forces imparted by closing the jaws of the clip during an initial deployment and/or tension/compression forces applied during reopening and reclosing of the jaws of the clip during any subsequent reopening and repositioning of the clip, while permitting the decoupling and final deployment of the clip by application of a relatively low release force.

Exemplary embodiments of the present disclosure comprise a clip mountable over a distal end of an endoscope via an adapter and releasably coupled to extending members so that, prior to final deployment, the clip may be moved relative to the adapter between an insertion configuration, an initial deployment configuration, and a review configuration. In the review configuration, the clip is moved distally away from the endoscope (or the endoscope is withdrawn proximally away from the initially deployed clip) to enhance the user’s view of the clip and the clipped tissue via the endoscopic viewing system prior to final deployment of the clip.

In an exemplary embodiment, a distal end of each of the extending members is fixed to a distal coupling releasably coupled to the clip via a release member. In various exemplary embodiments, the release member comprises a thin longitudinal member, such as a wire, a tube, a string, or a suture, which can be translated longitudinally relative to the extending member. In an exemplary embodiment, the release member can be transitioned from a retaining position in which a retaining portion of the release member (i.e., a distal portion thereof) blocks a part of the clip (i.e., a side portion thereof) from decoupling from the distal coupling to a release position in which the retaining portion of the release member is withdrawn from the blocking position and the clip is permitted to decouple from the distal coupling. It will be understood by those of skill in the art that terms proximal and distal, as used herein, refer to a direction toward and away from, respectively, a user of the device (e.g., along a longitudinal axis of the device and/or the endoscope or other insertion device). Thus, the proximal end of the device remains outside the body accessible to a user while, in use, the distal end of the device is inserted into the body adjacent to a target portion of internal bodily tissue to be treated.

In an exemplary embodiment, each of the distal couplings is sized, shaped and configured to couple to a respective one of the jaws of the clip, e.g., where the clip has two jaws on opposing sides of the clip, each of the distal couplings may also be coupled to a portion of its respective jaw that is opposed to the portion of the other jaw to which the other distal coupling is coupled. In an exemplary embodiment, each jaw includes an opening through which a respective distal coupling can partially extend. Each of the openings is formed through a side of the jaw opposite a side of the jaw configured to contact the gripped tissue (e.g., opposite the teeth of the jaw), with a small side portion of the jaw remaining between the opening and the edge of this side of the jaw. The distal couplings of one embodiment are generally cylindrical with each having a channel extending longitudinally therethrough and at least one recess formed in a side of the cylinder. In an embodiment, the channel in the distal coupling is sized, shaped, and configured for the release member to extend therethrough. In an embodiment, the recess is sized, shaped, and configured for the side portion of the jaw to be received therein. This side portion of jaw can be retained in the recess by the release member, e.g., in a frictional coupling, to be described in greater detail below.

The recess of each of the distal couplings is sized so that the side portion of the jaw can move therewithin while the release member continuously retains the side portion of jaw within the recess. The movement of the side portion of the jaw includes rotation and translation relative to the distal couplings and/or the extending members as the clip transitions between an open configuration (e.g., insertion configuration) in which the jaws are open and a closed configuration (e.g., deployment configuration) in which the jaws are closed. In some cases, the torsional forces involved in opening and closing the jaws can force the side portion of the jaw against the retaining portion of the release member blocking the side portion of the jaw from exiting the recess. The release member is configured to withstand these forces during positioning and potential reopening/repositioning of the clip while permitting the removal of the release members by a relatively low actuation force when the user is ready to finally deploy the clip.

When the user desires to release (e.g., fully deploy) the clip, each of the release members is translated so that the retaining portion of the release member is withdrawn from a window providing access into and out of the recess of the distal coupling to remove the retaining force. In some embodiments, the release member is withdrawn proximally through the channel of the distal coupling to remove the retaining force. In some embodiments, the release member comprises a hook-shaped distal end that wraps around the side portion of the jaw to provide the retaining force and, in these embodiments, the release member is advanced distally to remove the retaining force.

In an embodiment, the recess is defined by a surface shaped so that, after the release member is withdrawn, the distal couplings can be withdrawn proximally relative to the clip via the extending members. In an embodiment, the surface is an angled surface that urges the distal couplings to rotate when the distal couplings are brought into abutting contact with the side portion of the jaw and a proximal force is applied to the distal couplings via the extending members, e.g., after the user determines that the target tissue has been clipped as desired and the user is ready to release (finally deploy) the clip. At this point, with the clip fixed to the target tissue as desired, the user first withdraws the release members relative to the distal couplings such that side portions of each of the jaws remains within the corresponding recess but with these side portions of the jaws being permitted to move outside the recesses. In an embodiment, withdrawing the extending members proximally brings the angled surfaces of each of the distal couplings into contact with the side portion of its respective jaw, causing the distal couplings to rotate to permit further proximal movement, such that the distal couplings are drawn through the openings in the jaws and the clip is fully decoupled from the proximal couplings and from the remainder of the clipping system. In other embodiments, the distal surface of the recess is not angled.

In an exemplary embodiment, the release members extend from a proximal end accessible by an operating physician to a distal end that, in the retaining position, couples to a clip and/or couplings of the extending members. The distal ends of the release members in one embodiment are not anchored to the system. In an exemplary embodiment, a retaining portion of each of the release members, in the retaining position, extends fully through the channel of the distal coupling, e.g., the distal portion of the release members extend through a proximal end of the channel, wrap around the side portion of the jaw received within the recess and extend out the distal end of the distal coupling to prevent the side portion of the jaw from exiting the recess. In an exemplary embodiment, the retaining portion of the release members comprises a hook shape that, in the retaining position, wraps around both sides of the side portion.

A distal portion of the release members may curve around the distal end of the distal couplings and/or around the side portion of the jaw. This configuration functions to retain the side portion of each side of the jaw within a corresponding one of the recesses, e.g., by blocking the exit to the recess, preventing the side portion of the jaw from moving out of the recess, while permitting the jaw a desired amount of rotation and/or translation within the recess. Accordingly, the jaws can transition between the open configuration (insertion configuration) and the closed configuration (deployment configuration) while the release members remain in the retaining position holding the side portions of the jaws in place within their respective recesses. The release member is structured so that rotational and/or translational forces applied by the side portion of each jaw against the corresponding release member does not break the distal portion of the release member, withdraw it from the recess, or in any other way move the release member relative to the jaw to free the clip during one or multiple successive opening and closing operations as will be described below.

The release members, distal couplings and the side portion of the clip are constructed so that frictional forces between the release members and the distal couplings are sufficient to withstand lateral forces and/or torsional forces applied to the release members during movement of the clip on and off of the cap (e.g., during a repositioning of a previously placed clip). As will be described below in more detail, to re-open and reposition the clip, the spring forces urging the clip toward the closed position must be overcome as the clip is drawn proximally back over the cap forcing the jaws of the clip open. The frictional engagement of the release members with inner surfaces of the distal couplings must be sufficient to maintain the release members engaged with the distal couplings throughout this procedure so that the clip remains coupled to the apparatus for repositioning and re-deployment as many times as is needed by the user to capture the target tissue in the desired manner. Thus, radially outwardly directed forces as the clip is re-opened (in opposition to a natural bias or spring force urging the clip to a closed position) is opposed by the stiffness of the release members and the frictional engagement of the release members with the distal couplings.

When it is desired to release the clip, the distal portion of the release members is extended distally or retracted proximally from the retaining position blocking the window of the recess, overcoming the frictional forces retaining the clip, to free the jaws from the distal couplings so that the clip is finally separated from the rest of the apparatus. In an embodiment, the release member is translated proximally relative to the distal couplings until the distal portions of the release members are no longer blocking the window to the recess. In an embodiment, a hook-shaped distal portion of the release member is translated distally until the distal portion is no longer blocking the window to the recess.

In various exemplary embodiments, the release member is sized, shaped, and formed from a material having properties suitable for the above-described purposes as will be understood by those skilled in the art. In general, determining the size, shape and material for the release members involves a balance between properties including the strength, stiffness, flexibility, resilience, and frictional properties of the release members as they engage the distal couplings. In an exemplary embodiment, the release members are formed of Nitinol wire and the distal couplings are formed of stainless steel. However, those skilled in the art will understand that other materials may be used.

FIGS. 1-9 show a clipping system 100 for treating tissue defects and/or perforations according to an exemplary embodiment. The system comprises a clip 110 configured to be inserted through, for example, a body lumen to a target area to clip target tissue. The clip 110 is insertable to the target area via an insertion device navigated to a target location within a living body (e.g., passing through natural bodily lumens) using, for example, an endoscope (not shown) as would be understood by those skilled in the art.

As shown in FIG. 1, the clip 110 is coupled to an adapter 150 (formed, for example, as a cap of transparent material)), which is configured to be mounted over the distal end of the endoscope (e.g., with a friction fit) so that the vision system and working channel(s) of the endoscope are unimpaired and so that the working channel(s) and the vision system are aimed into an open lumen of the adapter 150. The clip 110 is configured to be moved proximally and distally over the adapter 150 via extending members 120, to which the clip 110 is releasably coupled via distal couplings 130 and release members 140 (the release members 140 are shown in greater detail in FIGS. 5-6). The clip 110 is movable between an insertion configuration and an initial deployment configuration and between the initial deployment configuration and a review configuration and from the review configuration back to the insertion configuration. In FIG. 1, the clip 110 is in the review configuration, as will be described in greater detail below.

In operation, the adapter 150 is mounted over the distal end of the endoscope and the endoscope (with the clip 110 and adapter 150) is navigated to a target site with the clip 110 in the insertion configuration. In the insertion configuration, the clip 110 is mounted over the adapter 150 with the jaws 111 spread apart from one another so that tissue drawn into the lumen of the adapter 150 is positioned between the jaws 111. When a target portion of tissue has been drawn into the lumen of the adapter 150, the user may move the clip 110 from the insertion configuration toward the initial deployment configuration, by moving the extending members 120 distally relative to the adapter 150 in a known manner (e.g., using one or more actuators on a handle which is not shown), pushing the clip 110 distally off of the adapter 150 so that a natural bias of the clip 110 forces the jaws 111 to a closed configuration, in which the jaws 111 are moved toward one another to grip the tissue that was previously drawn into the adapter 150 (e.g., by suction, grasper, etc., when the clip 110 was in the insertion configuration).

After tissue has been preliminarily clipped by the jaws 111 in an initial deployment configuration, the endoscope may be moved proximally relative to the clip 110 to the review configuration by withdrawing the endoscope proximally while extending the extending members 120 distally so the clip 110 remains clipped to the tissue while the increased distance of the endoscope and the adapter 150 from the clip 110 enhances the user’s ability to observe the position of the clip 110 by widening the field of view of the endoscopic vision system while also allowing for movement of the endoscope relative to the clip 110 to enable more extensive observation of the placement and/or position of the clip 110 relative to the target tissue from different viewing angles.

If the user determines the position of the clip 110 is incorrect or sub-optimal, the user may move the endoscope distally to position the distal end of the adapter 150 adjacent to the clip 110 by sliding the endoscope distally over the extending members 120. The extending members 120 may then be withdrawn proximally while holding the endoscope in position to retract the clip 110 into contact with the distal end 152 of the adapter 150. Pulling the clip 110 against the tapered distal end 152 forces the clip 110 to open releasing the previously clipped tissue and permitting the clip 110 to return to the insertion configuration with the jaws111 spread apart from one another stretched over the adapter 150. The user may then reposition the endoscope and the clip 110 and repeat these steps to adjust the placement and/or position of the clip 110 relative to the target site so that the clip 110 will be finally deployed only when the placement of the clip 110 satisfies the user. That is, when the user determines in the review configuration that the clip 110 is not positioned as desired, the clip 110 may be re-opened and removed from the tissue so that the device can be re-positioned until the clip 110 is closed over the target portion of tissue as desired.

When the user is satisfied that the clip 110 is positioned as desired, the clip 110 may be moved from the initial deployment configuration to a final deployed configuration in which the clip 110 is separated from the rest of the clipping system 100 so that the clip 110 may be left in place clipped over desired tissue while the rest of the clipping system 100 is withdrawn from the body.

In an exemplary embodiment, as shown in detail in FIGS. 3-6, a distal coupling 130 is fixed to the distal end 122 of each of the extending members 120. Each of the distal couplings 130 is releasably coupled to a corresponding jaw 111 of the clip 110 via a corresponding one of the release members 140. As indicated above, the release members 140 can be withdrawn proximally relative to their corresponding distal couplings 130 by application of a proximal force to the release members 140 relative to the extending members 120 that exceeds a predetermined threshold value (i.e., the force required to overcome frictional forces between the release members 140, the distal couplings 130 and the jaws 111) to pull the release members 140 out of the distal part of the distal couplings 130.

As indicated in more detail below, a portion of each of the jaws 111 is held within the first recess 134 of the distal coupling 130 by the corresponding one of the release members 140 which extends across a window 137 of the recess so that the portion of the jaw 111 is held therein. Thus, withdrawal of the release members 140 from the distal ends 132 of the distal couplings 130 permits the portions of the jaw 111 to move out of the first recesses 134 through the window 137 so that the clip 110 is separated from the distal couplings 130 and from the rest of the clipping system 100 which may then be withdrawn from the body leaving the clip 110 in place. This fully deploys the clip 110.

While the retaining force of the release members 140 remains in place, however, longitudinal movement of the extending members 120 proximally and distally relative to the adapter 150 and the endoscope move the clip 110 between the insertion configuration, the initial deployment configuration, and the review configuration as often as required by the user. In these operations, the extending members 120 and the release members 140 are moved together with one another (e.g., by operation of a common actuator). In the final deployment, the release members 140 are moved relative to the extending members 120 and the distal couplings 130.

As shown in FIG. 2, the clip 110 includes two jaws 111 connected to one another via hinges 117. In one embodiment, each of the jaws 111 extends along a curve from a first end 112 to a second end 113 so that a first one of the hinges 117 connects the first ends 112 of each of the jaws 111 to one another, while a second one of the hinges 117 connects the second ends 113 of each of the jaws 111 to one another. In an embodiment, the hinges 117 are spring biased, biasing the jaws 111 toward the closed configuration (deployment configuration) in which gripping features 114 of the jaws 111 are moved toward one another. Each of the jaws 111 of this embodiment includes gripping features 114 to enhance a hold of the clip 110 on the clipped tissue. In the present embodiment, the gripping features 114 comprise teeth sized and shaped so that, in the deployment configuration, the teeth of one of the jaws 111 intermesh with the teeth of the other jaw 111. In particular, in the deployment configuration, the jaws 111 extend toward one another so that the target tissue may be gripped between the jaws 111 via the gripping features 114.

However, when the clip 110 is mounted over the adapter 150 in the insertion configuration, the jaws 111 extend about opposing portions of the adapter 150 so that an exterior surface of the adapter 150, e.g., a distal portion 156 thereof, maintains the clip 110 in an open configuration, with the jaws 111 separated from one another, as shown in FIG. 9. Thus, when the clip 110 is mounted over the adapter 150, target tissue may be drawn into a lumen 153 in the adapter 150 so that it is received between the jaws 111. When the clip 110 is moved distally off of the adapter 150, the clip 110 closes under the natural bias of the hinges 117 over the tissue that had been drawn into the adapter 150.

It will be understood by those of skill in the art that the hinges 117 and/or jaws 111 of the clip 110 may be formed of any of a variety of materials so long as the hinges 117 bias the jaws 111 toward the deployment configuration, as described above, and so that the bias is sufficiently strong to maintain the clip 110 in clipped position over target tissue after the clip has been finally deployed. In one example, portions of the clip 110 (e.g., the hinges 117) are formed of a shape memory alloy such as, for example, Nitinol to provide and/or add to the bias toward the closed configuration. Furthermore, in an alternative embodiment, the jaws 111 are not biased toward the closed position and are closed by any other known mechanism. The release members, extending members and distal couplings will operate in this embodiment in the same manner to move the clip between the insertion, initial deployed, review and finally deployed configurations, as would be understood by those skilled in the art.

According to an exemplary embodiment, each of the jaws 111 includes an opening 115 extending through the clip 110 from a first surface 118 of the clip 110 facing the adapter 150 (in the insertion configuration) to a second surface 119 of the clip 110 facing away from the adapter 150. In one embodiment, the opening 115 of each of the jaws 111 is sized, shaped, and configured to receive a portion of a distal coupling 130 therein. The distal couplings 130 are coupled to the jaws 111 via the release members 140, each of which encloses a side portion 116 of a corresponding one of the jaws 111 within a recess (e.g., a first recess 134) and maintains a first position (retaining position) thereof via frictional forces between the release members 140, surfaces of the distal couplings 130 (e.g., inner surfaces thereof) and the side portion 116 of the jaws 111 (e.g., an outer surface thereof), to be described in greater detail below.

The distal couplings 130 can translate and rotate relative to the jaws 111 while remaining coupled thereto when the release members 140 are in the retaining position. In an embodiment, the opening 115 extends through each of the jaws 111 midway between the first and the second ends 112, 113 so that the openings 115 extend through opposing portions of the clip 102. In an embodiment, the openings 115 are located adjacent to a side 170 of the jaw 111 opposing the gripping features 114, wherein the side portion 116 of the jaw 111 is defined as the portion of the jaw 111 between the opening 115 and the side 170.

As shown in FIGS. 3-6, a distal coupling 130 is attached at a proximal end 131 to a distal end 122 of a corresponding one of the extending members 120. In an embodiment, the distal coupling 130 and the extending member 120 are rigidly fixed to one another, e.g., welded, so that longitudinal motion of the extending member 120 translates to longitudinal motion of the corresponding distal coupling 130. In an embodiment, the distal coupling 130 is cylindrical with a channel 133 extending fully through the distal coupling 130 from the proximal end 131 to a distal end 132. The wall thickness of the distal coupling 130 of this embodiment is selected so that the distal coupling 130 possesses a minimum strength that can withstand forces applied thereon during the exemplary operations without deformation. In an embodiment, a first recess 134 is formed in the side of the distal coupling 130. In an embodiment, the first recess 134 is laser-cut into the distal coupling 130. In an embodiment, a distal surface 135 defining the first recess 134 is angled to facilitate the decoupling of the distal coupling 130 from the clip 110 in the final deployment, to be described in further detail below.

In an embodiment, the distal coupling 130 further includes a second recess 136 proximal to the first recess 134. The second recess 136 facilitates the threading of the release members 140 through the channel 133 of the distal coupling 130 and around the side portion 116 of the clip 110 during assembly. It should be understood that, in some embodiments, the second recess 136 is not required and the release members 140 can be passed through the channel 133 and around side portion 116 of the clip 110 without the aid of the second recess 136.

During assembly, the distal couplings 130 are passed partially through the openings 115 in the jaws 111 of the clip 110 so that the side portion 116 of each of the jaws 111 is received in the corresponding first recess 134. The release members 140 are then passed through the channels 133 of the distal couplings 130, around the side portions 116 of the clip 110, and past the distal end 132 of the distal couplings 130, as shown in FIG. 6. Thus, the release members 140 act as a wall, preventing the side portions 116 of the jaws 111 from exiting the first recesses 134 via the window 137. To enable the removal of the wall, the distal ends 142 of the release members 140 are not fixed to the distal couplings 130, the clip 110 or any other part of the insertable portion of the clipping system 100. In other words, the distal end 142 of each release member 140 is unanchored permitting the release members 140 to move relative to the distal couplings 130 and the clip 110 so that the clip 110 can be finally deployed.

As described above, the release members 140 can be transitioned between a retaining position and a release position relative to the distal couplings 130. In the retaining position, the release members 140 extend through the channels 133 of the distal couplings 130, around the side portions 116 of the clip 110, and past the distal end 132 of the distal couplings 130. In an embodiment, a proximal portion 143 of each release member 140 extends from the proximal end 141 of the release members 140 to the proximal end 131 of the distal couplings 130 while the distal portion of each release member 140 extends, when the release members 140 are in the retaining position, to the distal end 132 of the distal coupling 130 or projects distally out of the distal end 132 by any desired length.

In an embodiment, a distal portion 144 of the release members 140 extends, in the retaining position, from the proximal end 131 of the distal coupling 130 through the distal coupling 130 to the distal end 132 of the distal coupling. In an embodiment, a curved distal part 145 of the release member 140 extends from the distal end of the retaining distal portion 144 distally out of the distal end 132 of the distal couplings 130 to the distal end 142 of the release member 140. In an embodiment, a retaining portion 146 of the release member 140 extends across the first recess 134 of the distal coupling 130 to block the side portion 116 of the clip 110 from exiting the first recess 134 through the window 137.

In an embodiment, at least the distal portion 144 of the release members 140 (including the retaining portion 146 and the curved distal part 145) is formed of a shape memory alloy such as Nitinol. In particular, the distal part 145 can be formed so that it is biased toward a curved shape. As would be understood by those skilled in the art, the curved shape of the distal part 145 functions to prevent unwanted longitudinal movement of the release members 140 relative to the distal couplings 130 during transitions of the clip between the insertion configuration, the initial deployment configuration, and the review configuration. Accordingly, the coupling between the clip 110, the distal coupling 130 and the release member 140 is configured such that the release member 140 maintains a retaining position (e.g., the retaining portion 146 provides the wall preventing the side portion 116 of the jaw 111 from exiting the first recess 134 through the window 137 of the distal coupling 130) throughout all operations of the clipping system 100 prior to the final deployment.

However, the distal portion 144 remains sufficiently flexible such that, when it is desired by a user to release the clip 110, a proximal force applied to the release members 140 relative to the extending members 120 overcomes the bias of the distal part 145 such that the distal part 145 can bend (e.g., straighten out) and pass proximally through the distal end 132 of the distal coupling 130 to release the clip 110. It is noted that the entirety of the distal portion 144 can be formed of the same shape memory alloy (e.g., Nitinol), which can be selected for properties such as stiffness and flexibility.

In an embodiment, the proximal portion 143 of each of the release members 140 may also be formed of the same shape memory alloy as the distal portion 144 such that the release members 140 can comprise respective unitary members. In other embodiments, the proximal portion 143 may be formed of a different material. For example, it may not be necessary for the proximal portion 143 to possess properties (stiffness, flexibility, etc.) similar to the distal portion 144. In an embodiment, the proximal portion 143 may be formed of stainless steel.

It is noted that a shape-memory alloy such as Nitinol can possess properties beneficial to the operations of the release member 140 described above. A Nitinol wire is stiffer relative to many other biocompatible alloys, such as stainless steel, and can resist deformation that could otherwise occur during use. For example, if a less stiff alloy were used for the release members 140, notches or cracks may form in the wire due to the forces imparted by the clip. Additionally, Nitinol has a flexibility that permits the bending of the wire through the desired range of motion without resulting in plastic or permanent deformation.

In other embodiments, the release members 140 can comprise different materials and configurations. For example, the release members 140 can comprise wires of different materials, or hypotube. In some embodiments, the release member 140 can comprise a suture, or some other type of string or thread. The type of release member 140 can be selected in view of various performance considerations including friction, stiffness, and flexibility properties.

In the retaining position, the release members 140 function to retain the side portions 116 of the jaws 111 within the first recess 134 of the distal couplings 130 by blocking the window 137 and preventing the side portions 116 from moving out of the first recesses 134. However, the release members 140 allow the side portions 116 to translate and rotate within the first recess 134 as the jaws 111 are opened and closed while resisting the torsional forces imparted during these operations. Accordingly, the jaws 111 can transition between the open configuration (insertion configuration) and the closed configuration (deployment configuration) while the release members 140 remain in the retaining position. Any rotational or translational forces of the side portion 116 of the jaw 111 against the release member 140 will not cause the distal portion 144 of the release member 140 to break, withdraw, or otherwise release the clip 110 during one or multiple successive opening and closing operations.

When it is desired to release the clip 110, the release members 140 can be retracted proximally relative to the distal couplings 130, bringing the distal part 145 of the release members 140 proximally through at least part of the channel 133 of the distal couplings 130 to reduce or remove the retaining force, thus permitting the distal couplings 130 to decouple from the jaws 111. In particular, the frictional forces between the release members 140, the distal couplings 130 and the clip 110 can be overcome by a proximal force on the release members 140 such that the distal part 145 of the release member 140 can bend (e.g., straighten) and pass through the distal end 132 of the distal coupling 130 through the channel 133 to a release position.

With the retaining force removed, the side portions 116 of the jaws 111 are permitted to exit the first recess 134 of the distal coupling 130 via the window 137, which is facilitated by the angled surface 135 in the distal coupling. With the clip 110 closed over target tissue, the distal couplings 130 can be withdrawn proximally relative to the clip 110 via the extending members 120. When the angled surface 135 is brought into abutting contact with the side portion 116 of the jaw 111 the distal couplings 130 are urged to rotate to permit further proximal motion, such that further proximal force draws the distal couplings 130 through the opening 115 in the jaws 111. When the distal end 132 of the distal couplings 130 passes through the opening 115 the clip 110 is fully decoupled from the system in a final deployment.

It is noted that, in other embodiments, the distal coupling 130 can be longitudinally mirrored relative to the embodiments described above, e.g., with the first recess 134 facing the clip 110 and the distal coupling 130 substantially outside the clip 110. In these embodiments, withdrawing the distal couplings 130 proximally allows the clip 110 to decouple from the distal couplings 130 without requiring the distal coupling 130 to pass through the opening 115 in the jaws 111.

As shown in FIG. 7, the adapter 150 extends from a proximal end 151 to a distal end 152 and includes a lumen 153 extending therethrough. A proximal portion 154 of the adapter 150 is configured to be mounted over the distal end of the endoscope while a distal portion 156 of the adapter 150 is configured to receive the clip 110 thereover in the insertion configuration. The proximal portion 154 of the adapter 150 may be mounted to the endoscope via, for example, a friction fit, so that the lumen 153 of the adapter 150 is substantially longitudinally aligned with a distal end of the endoscope. Thus, tissue may be viewed through the lumen 153 via an optical system of the endoscope and suction and/or tools may be applied through the adapter 150 via the working channel of the endoscope. In another embodiment, to enhance the visibility of the tissue and/or the clip 110, the adapter 150 is formed of a transparent material.

In one embodiment, the proximal portion 154 of the adapter 150 includes a pair of longitudinal holes 155 extending longitudinally through a wall thereof. Each of the longitudinal holes 155 is configured to slidably receive a corresponding one of the extending members 120, which extend through the longitudinal holes 155 to be coupled to the clip 110 via the distal couplings 130.

An outer diameter of the distal portion 156 of the adapter 150 is sized, shaped, and configured to receive the clip 110 thereover when the jaws 111 are stretched apart from one another in the insertion configuration. In an exemplary embodiment, the distal portion 156 tapers toward the distal end 152 so that the bias of the clip 110 urging the clip 110 closed aids in moving the clip 110 distally off of the adapter 150 to assume the deployment configuration. When the clip 110 is mounted on the distal portion 156 of the adapter 150, with the jaws 111 extending over opposing portions of the adapter 150, the exterior surface of the adapter 150 pushes the jaws 111 of the clip 110 radially outward maintaining the jaws 111 open in the insertion configuration (i.e., the jaws 111 are held open against their natural bias by the adapter 150).

The clip 110 of this embodiment remains mounted over the adapter 150 in the open insertion configuration so long as a sufficient proximally directed tension is applied to the extending members 120 to hold the clip 110 in place. When this tension is removed from the extending members 120, or the extending members 120 are extended distally relative to the adapter 150, the natural bias of the clip 110 draws the jaws 111 toward one another over the taper of the distal portion 156 pushing the clip 110 distally over the adapter 150 until the clip 110 slides distally off of the adapter 150. When the jaws 111 move distally past the distal end of the adapter 150, they are freed to close under their natural bias clipping any tissue that had been drawn into the adapter 150. As would be understood by those skilled in the art, the taper of the adapter 150 also aids in forcing the jaws 111 apart from one another as the clip 110 is drawn proximally back over the adapter 150 (e.g., when the user wants to remove a clip 110 from an initial deployed position clipped to tissue).

In an embodiment, the distal portion 156 of the adapter 150 includes longitudinal grooves 157 to facilitate transitioning the device between the insertion state and the initial deployment state and between the initial deployment state and the review state, e.g., by providing a support channel for the distal coupling 130 (when the device is in the insertion state, transitioning out of the insertion state into the deployment state, or transitioning into the insertion state from the deployment state) and/or for the extending members 120 (when the distal coupling 130 is moved distally past the longitudinal groove 157), e.g., to help prevent kinking.

In some embodiments, the distal portion 156 of the adapter 150 includes longitudinally extending flat portions distributed about the circumference of the exterior surface of the adapter 150 to reduce friction between the clip 110 and the distal portion 156 of the adapter 150 to facilitate movement of the clip 110 between the insertion and initial deployed configurations. In some embodiments, the distal portion 156 of the adapter 150 includes projections extending radially into the channel of the adapter 150 at its distal end 152. The projections can be angled with respect to a longitudinal axis of the adapter 150 so that, when the clip 110 is drawn proximally from the initial deployed configuration until the jaws 111 abut the distal face, the angles of these projections act as ramps to facilitate the re-opening of the jaws 111 against their natural bias, toward the open insertion configuration.

As shown in FIG. 1 and in greater detail in FIG. 8, the clipping system 100 further includes a longitudinal member 161 through which the release members 140 and the extending members 120 extend coextensively. The longitudinal member 161 extends from a proximal end 162 to a distal end 163 and can extend coextensively with the insertion device, e.g., endoscope. The release members 140 extend from proximal ends 141 and the extending members 120 extend from proximal ends 121 that are accessible by a user, e.g., an operating physician, at the proximal end 162 of the longitudinal member 161. The clipping system 100 further includes two medial members 164 that branch off the distal end 163 of the longitudinal member 161, each of the medial members 164 being configured to a receive a respective pair of one of the extending members 120 and one of the release members 140, e.g., a first one of the extending members 120 and a first one of the release members 140 extends through a first one of the medial members 164 and a second one of the extending members 120 and a second one of the release members 140 extends through a second one of the medial members 164. Each of the medial members 164 extends from a proximal end 165 at the distal end 163 of the longitudinal member 161 to respective distal ends 166 on opposing sides of the proximal portion 154 of the adapter 150.

The longitudinal member 161 and the medial members 164 each have channels extending therethrough through which the extending members 120 and the release members 140 extend. At the distal ends 166 of the medial members 164, a respective extending member 120 and a release member 140 can pass through longitudinal holes 155 in the proximal portion 154 of the adapter 150. Accordingly, the release members 140 and the extending members 120 extend from their proximal ends 141, 121, coextensively through the longitudinal member 161 and, at the distal end 163 of the longitudinal member 161, the first one of the release members 140 and the first one of the extending members 120 split from the second of the release members 140 and the second one of the extending members 120, the first of the release members 140 and the first one of the extending members 120 running through the first one of the medial members 164 and the second one of the release members 140 and the second one of the extending members 120 running through the second one of the medial members 164. The first one of the release members 140 and the first one of the extending members 120 then run through a first one of the longitudinal holes 155 in the proximal portion 154 of the adapter 150 and the second one of the release members 140 and the second one of the extending members 120 then run through a second one of the longitudinal holes 155 in the proximal portion 154 of the adapter 150.

As discussed above, the clip 110 is mounted to the insertion device, which may be, e.g., any standard endoscope. The clip 110 in this embodiment is mounted to the endoscope via the adapter 150, which is sized, shaped and configured to be mounted over the distal end of the endoscope. In an embodiment, the clipping system 100 further includes an endoscope clip 167 including a rounded surface sized, shaped, and configured to clip onto the exterior surface of the endoscope. As will be understood by those of skill in the art, the endoscope is configured to be inserted through a body lumen to a target area within the lumen and thus, must be sufficiently flexible to navigate through even tortuous paths of the body lumen.

In an alternative embodiment, a clipping system can include a coupling mechanism comprising nested tubes (e.g., hypotubes). Relative to the clipping system 100 described above, in which the coupling mechanism comprises the distal coupling 130 and the release member 140, the coupling mechanism of these embodiments comprises an outer tube and an inner tube in which the inner tube is slidably coupled to the outer tube, e.g., an outer diameter of the inner tube corresponds to an inner diameter of the outer tube. The outer tube includes a recess formed in its side, e.g., extending radially inward transversely to the longitudinal axis of the outer tube, comparable to the first recess 134 of the distal coupling 130 of the clipping system 100. The inner tube includes a recess formed in its distal end, e.g., extending longitudinally inward, that functions similarly to the release member 140 of the clipping system 100, e.g., retains the clip in a retention position and can be retracted proximally to release the clip. To assemble this exemplary clipping system, a portion of a clip can be received in the recess of the outer tube, then the inner tube can be passed distally into a proximal end of the lumen of the outer tube such that the portion of the clip is received in the recess of the inner tube, forming a retention configuration that prevents the portion of the clip from exiting the recess(es).

FIGS. 10-11 shows a clipping system 200 including a coupling mechanism for a clip comprising nested tubes according to various exemplary embodiments. It should be understood that some aspects of the clipping system are similar to the clipping system 100 described above. In particular, the clip of the clipping system 200 may be similar to the clip 110 of the clipping system 100. FIGS. 10-11 show a portion 230 of a clip that corresponds to the side portion 116 of the clip 110 of the clipping system 100.

The clipping system 200 comprises an outer tube 210 and an inner tube 220. The outer tube 210 is configured similarly to the distal coupling 130 of the clipping system 100. The outer tube 210 is attached (e.g., rigidly fixed) at a proximal end 211 to a distal end of an extending member (not shown). The outer tube 210 is cylindrical with a channel 213 extending through the outer tube 210 from the proximal end 211 to a distal end 212, as shown in FIG. 11. In an embodiment, a recess 214 is formed in the side of the outer tube 210, e.g., laser-cut. A distal surface 215 defining the recess 214 is angled to facilitate the decoupling of the outer tube 210 from the clip, similar to the first recess 134 of the clip 110. A window 216 provides access to the recess 214.

The inner tube 220 is attached at its proximal end 221 to a cable 225 that can be translated longitudinally such that the inner tube 220 can translate relative to the outer tube 210. The inner tube 220 includes a recess 223 formed in its distal end 222. During assembly, the portion 230 of the clip is received in recess 214 of the outer tube 210. The inner tube 220 is then advanced distally through the proximal end 211 of the outer tube 210 and through the channel 213 until the portion 230 of the clip is received in the recess 223 of the inner tube 220 and the distal end 222 of the inner tube 220 is distal to the recess 214 of the outer tube 210, as shown in FIG. 11. Thus, a retaining portion 224 of the inner tube 220 provides a wall blocking the window 216, preventing the portion 230 of the clip from exiting the recess 214 of the outer tube 210.

Similar to the clipping system 100, the coupling mechanism of the clipping system 200 permits the portion 230 of the clip to translate and rotate within the recess 214. The inner tube 220 can be transitioned between a retaining position and a release position relative to the outer tube 210 by the cable 225. When it is desired to release the clip, the inner tube 220 is retracted proximally relative to the outer tube 210 to remove the retaining force and permit the outer tube 210 to decouple from the clip, similar to the clipping system 100.

FIGS. 12-20 show a clipping system 300 for treating tissue defects and/or perforations according to another exemplary embodiment. It should be understood that some aspects of the clipping system 300 are similar to the clipping system 100 described above. Aspects of the clipping system 300 including extending members 330 (shown in, e.g., FIG. 17), an adapter (not shown), and additional proximal components (medial members, endoscope clip, etc.) (not shown) may be similar to the extending members 120, the adapter 150 and additional proximal components of the clipping system 100 described above. The clipping system 300 comprises a clip 310 and a coupling mechanism on each side of the clip 310 including a distal coupling 340 and a release member 360 according to an alternative embodiment in which the release member 360 is pushed distally to transition the release member 360 from the retaining configuration to the release configuration to permit the decoupling of the clip 310 from the rest of the clipping system 300.

Similar to the clipping system 100, the clip 310 of the clipping system 300 is configured to be moved proximally and distally over the adapter via the extending members 330, to which the clip 310 is releasably coupled via distal couplings 340 and release members 360, shown in detail in FIGS. 14-20. The clip 310 is movable between an insertion configuration, an initial deployment configuration and a review configuration. The operations of the clipping system 300 to transition between these deployment configurations are similar to those of the clipping system 100 described above with the release of the clip 310 differing from the previously described embodiments. When the user is satisfied that the clip 310 is positioned as desired, the clip 310 may be moved from the initial deployment configuration to a final deployed configuration in which the clip 310 is separated from the rest of the clipping system 300.

As shown in FIG. 12, the clip 310 includes two jaws 311 connected to one another via hinges 321. Each of the jaws 311 extends from a first end 312 to a second end 313. A first one of the hinges 321 connects the first end 312 of a first one of the jaws 311 to a second one of the jaws 311 and a second one of the hinges 321 connects the second end 313 of the first one of the jaws 311 to the second end 313 of the second one of the jaws 311. Similar to the clip 110, the hinges 321 of the clip 310 are spring biased, biasing the jaws 311 toward a closed configuration (e.g., a deployment configuration) in which gripping features 314 of the jaws 311 are moved toward one another. In the closed configuration, the jaws 311 extend toward one another so that the target tissue may be gripped between the jaws 311 via the gripping features 314. When the clip 310 is mounted over the adapter in the insertion configuration, the jaws 311 are stretched to spread apart from one another as they extend over opposing portions of the adapter so that an exterior surface of the adapter maintains the clip 310 in the open configuration. When the clip 310 is moved distally off of the adapter, the clip 310 closes under the natural bias of the hinges 321, e.g., over tissue that had been drawn into the lumen of the adapter.

As shown in greater detail in FIG. 13, each jaw 311 includes a side part 316 extending off a side 315 of the jaw 311 opposing the gripping features 314. Each side part 316 includes extending portions 317 projecting away from the corresponding jaw 311 and a side portion 318 connecting the extending portions 317 to form an opening 319. The openings 319 extend through the clip 310 from a first surface (not shown) of the clip 310 facing the adapter to a second surface 322 of the clip 310 facing away from the adapter. In one embodiment, the opening 319 of each of the jaws 311 is sized, shaped, and configured to receive a portion of a distal coupling 340 therein.

The distal couplings 340 are coupled to the jaws 311 via the release members 360, each of which releasably holds the side portion 318 of a corresponding one of the jaws 311 within a recess 345 of the distal coupling 340 and maintains a first position (retaining position) thereof (e.g., via frictional forces) between the release members 360, surfaces of the distal couplings 340 (e.g., inner surfaces thereof) and the side portion 318 of the jaws 311 (e.g., an inner surface and/or an outer surface thereof), to be described in greater detail below.

In an embodiment, the side portion 318 includes a groove 320 on each side thereof. The groove 320 is sized and shaped to guide the release member 360 relative to the jaw 311, in particular, the motion of the jaw 311 relative to a hook portion 365 of the release members 360, e.g., during operation of the clipping system 300, to be described in greater detail below.

The side portion 318 of the clip 310 functions as a connection point to the distal couplings 340, release members 360 and the remainder of the clipping system 300. Relative to the clip 110 of the clipping system 100, in which the opening 115 is through a body of the jaw 111 and the side portion 116 is substantially integral with the side 170 of the jaw 111 and reinforced by the jaw 111 along the side 170, the side portion 318 of the clip 310 of the clipping system 300 are extended off the sides 315 of the jaws 311. Considered another way, the jaws 311 of the clip 310 have a cut out portion adjacent to the opening 319, reducing the width of the body of each jaw. However, it is not necessary for the side portions 318 to project away from the body of the jaws 311. In an alternative embodiment, the openings 319 can be formed within the lateral portions of the jaws 311.

As shown in FIGS. 14-15, in an exemplary embodiment, each of the distal coupling 340 includes a tube portion 341 comprising a recess 345 and a cap portion 347 within the tube portion 341 at a distal end 343 thereof. As shown in FIGS. 16-20, the distal coupling 340 is releasably coupled to a corresponding jaw 311 of the clip 310 by a release member 360 that includes a distal portion 363 that includes a hook portion 365.

As will be described in detail below, in this exemplary embodiment, the coupling mechanism between the clip 310 and the remainder of the clipping system 300, e.g., the arrangement of the distal coupling 340, the release members 360 and the clip 310, comprises some differences relative the clipping system 100. In an exemplary embodiment, the recess 345 within which the side portion 318 of the jaw 311 is received, and a window 346 providing access to the recess 345 faces toward the clip 310 although this is not necessary and the arrangement of the window 346 can be reversed but this requires that the opening 319 be large enough to facilitate the pulling of the distal couplings 340 out of the opening after the hook portion 365 has been moved distally to free the window 346.

As the arrangement shown in FIGS. 12-20 allows the distal couplings to remain outside the window 346, this embodiment may provide a more reliable release of the clip 310. In an exemplary embodiment, the release members 360 comprise a hook portion 365 that, in the retaining position, curves around the side portion 318 of the jaw 311 (e.g., so that each of the release members 360 extends through the corresponding distal coupling 340 outside the side portion 318 and bends back so that the distal end of the hook portion 365 passes back across the window 346 inside the window 346 of the clip 310. Thus, the side portions 318 of the jaws 311 are coupled to the distal couplings 340.

In this embodiment, the release members 360 are pushed distally relative to the distal couplings 340 and the extending members 330 until the distal hook portion 365 is positioned distally of the recess 345 so that the window 346 is open and the jaws 311 are freed to exit the recesses 345 releasing the clip 310 from the remainder of the clipping system 300. In an exemplary embodiment, the distal coupling 340 comprises a cap portion 347 closing off the distal end of the distal coupling 340 so that the hook portions 365 of the release members 360 are prevented from extending distally out of the distal end of the distal coupling 340 during or after the release of the clip 310.

The distal coupling 340 comprises a tube portion 341 and a cap portion 347, as shown in FIGS. 14-15. The tube portion 341 is attached at a proximal end 342 to a distal end 331 of a corresponding one of the extending members 330, as shown in FIG. 17. The distal coupling 340 and the extending member 330 are rigidly fixed to one another, e.g., welded, so that longitudinal motion of the extending member 330 translates to longitudinal motion of the corresponding distal coupling 340. In an embodiment, the tube portion 341 is cylindrical with a channel 344 extending through the tube portion 341 from the proximal end 342 to a distal end 343. In an embodiment, a recess 345 is formed in the side of the tube portion 341, e.g., by laser-cutting. Relative to the distal coupling 130 of the clipping system 100, the recess 345 of the distal coupling 340 is not defined by an angled distal surface. To be described in greater detail below, the angled distal surface is not required in view of the decoupling mechanism of the clipping system 300 described in greater detail below.

In an embodiment, the tube portion 341 of the distal coupling 340 further includes a cap portion 347 on its proximal end 342 formed by, e.g., laser-cutting. The tube portion 341 is cut in this manner to reduce the weight of the distal coupling 340 while providing a surface to which the extending member 330 can be fixed and lateral support to the release members 360 extending through the channel 344. It should be understood that, in some embodiments, the cap portion 347 is not required and this proximal portion of the tube portion 341 can comprise an uncut tube.

In an embodiment, the cap portion 347 is fixed to the tube portion 341 within the channel 344 of the tube portion 341 at the distal end 343 thereof. In particular, a proximal end 348 of the cap portion 347 is inserted to a depth in the channel 344 of the tube portion 341 substantially adjacent to a distal end of the recess 345 and a distal end 349 of the cap portion 347 extends slightly past the distal end 343 of the tube portion 341. The cap portion 347 includes a slot 350 extending through the proximal end 348 to a depth adjacent to the distal end 349, and the distal end 349 of the cap portion 347 is closed. The slot 350 is sized and shaped for the distal portion 363 of the release members 360 to be received therein, e.g., to guide the distal portion 363 of the release members 360 when the release members 360 are advanced distally to withdraw the retaining force from the recess 345 of the distal coupling 340, while preventing the distal portion 363 of the release members 360 from extending out the distal end of the distal coupling 340, as will be described in greater detail below.

In an embodiment, the release member 360 includes a proximal portion 362 that extends longitudinally, e.g., within and along with the extending member 330, and a distal portion 363 comprising the curved portion 364 and a hook portion 365. The release members 360 can be moved in concert with the extending members 330, e.g., by a common actuator, such that the extending members 330, the release member 360 and the distal coupling 340 all move together, e.g., while transitioning the clip 310 between the various deployment states prior to final deployment. During these deployment operations, each of the release members 360 remains in a retaining position in which the hook portion 365 wraps around the side portion 318 of the jaw 311 and closes the window 346 preventing the side portion 318 from exiting the recess 345 of the distal coupling 340, as shown in greater detail below in FIGS. 18-20.

To release the clip, the release members 360 are moved distally relative to the extending members 330 to decouple the distal coupling 340 from the clip 310. The user moves the release members 360 distally relative to the distal couplings 340 to push the hook portion 365 until the end of the hook portion is distal of the window 346 freeing the side portion 318 of the jaw 311 to exit the recess 345. That is, the release members 360 are pushed distally within the distal couplings 340 until the ends of the hook portions 365 moves out of contact with the side portion 318 of the jaw 311 so that the distal couplings 340 are no longer coupled to the clip 310, permitting the side portions 318 to exit the recesses 345 separating the clip 310 from the clipping system 300 in a final deployment of the clip 310.

In an embodiment, a proximal portion 362 of each release member 360 extends from a proximal end (not pictured) of the release members 360 through a corresponding one of the extending members 330 to a part of the release member 360 adjacent to the distal end 331 of the extending member 330 (e.g., when the release member 360 is in the retaining position). In an embodiment, the distal portion 363 of each of the release members 360 includes the curved portion 364 bending away from a longitudinal axis L of the portion of the release member 360 proximal thereof. In this embodiment, the curved portion 364 is oriented so that a portion of the release member 360 immediately distal to the first curved portion extends parallel to and laterally offset from the longitudinal axis L and a hook portion 365 includes a curve that bends through approximately 180 degrees so that the end of the hook portion 365 extends along the longitudinal axis L back toward the proximal end of the curved portion 364. The hook portion 365 generally comprises a first part 366 extending distally from the curved portion 364, a second part 367 extending from the first part 366 and curving toward the clip 310 in a 180 degree arc, and a third part 368 extending from the second part 367 proximally. The third part 368 comprises the retaining portion of the release member 360 that, in the retaining position, blocks access to the window 346, preventing the side portion 318 from exiting the recess 345.

As shown in the semi-transparent views of FIGS. 18-20, the hook portion 365 of the release member 360 is sized and shaped to wrap around the side portion 318 of the jaw 311 when the release member 360 is in the retaining position. In this position, the first part 366 and the third part 368 of the hook portion 365 are received in the grooves 320 on opposing sides of the side portion 318. In this position, the third part 368 of the hook portion 365 extends across the recess 345 between the side portion 318 of the jaw 311 and the window 346. The release member 360 can be transitioned into the release position by advancing the release member 360 distally into the slot 350 of the cap portion 347 such that the third part 368 (retaining portion) is brought out of contact with the side portion 318 of the jaw 111.

As described above, in this exemplary embodiment, the distal coupling 340 has a recess 345 that faces toward the clip 310. Accordingly, the distal coupling 340 does not need to be passed through the opening 319 in the jaw 111 to assemble the clipping system 300 or to release the clip 310 from the remainder of the clipping system 300 for final deployment.

During assembly, the release members 360 are in the release position within the distal coupling 340. The side portion 318 of the jaw 311 is inserted through the window 346 into the recess 345 of the distal coupling 340, and the release members 360 are transitioned into the retaining position by withdrawing the release members 360 proximally relative to the distal coupling 340. Thus, the third parts 368 of the release members 360 form a wall, preventing the side portions 318 of the jaws 311 from exiting the recesses 345.

As indicated above, the release members 360 can be advanced distally relative to their corresponding distal couplings 340 by application of a distal force to the release members 360 relative to the extending members 330 that exceeds a predetermined threshold value (i.e., the force required to overcome frictional forces between the release members 360, the distal couplings 340 and the jaws 311) to push the third part 368 of the release members 360 out of the recess 345 of the distal couplings 340. Thus, withdrawal of the release members 360 from the position blocking the recess 345 of the distal couplings 340 permits the side portion 318 of the jaw 311 to move out of the recess 345 through the window 346 so that the clip 310 can be separated from the distal couplings 340 and from the rest of the clipping system 300 which may then be withdrawn from the body leaving the clip 310 in place. This fully deploys the clip 310.

While the retaining force of the release members 360 remains in place, however, longitudinal movement of the extending members 330 proximally and distally relative to the adapter and the endoscope move the clip 310 between the insertion configuration, the initial deployment configuration, and the review configuration as often as required by the user. In these operations, the extending members 330 and the release members 360 are moved together with one another (e.g., by operation of a common actuator). In the final deployment, the release members 360 are moved relative to the extending members 330 and the distal couplings 340.

The distal couplings 340 can translate and rotate relative to the jaws 111 while remaining coupled thereto when the release members 360 are in the retaining position. The coupling between the clip 310, the distal coupling 340 and the release member 360 is configured such that the release member 360 maintains a retaining position (e.g., provides the wall preventing the side portion 318 of the jaw 311 from exiting the recess 345 of the distal coupling 340) throughout all operations of the clipping system 300 prior to the final deployment.

Similar to the release members 140 of the clipping system 100, all or part of the release members 360 of the clipping system 300 may be formed from a shape memory alloy (e.g., Nitinol), which can be selected for properties such as stiffness and flexibility. In other embodiments, all or part of the release members 360 may be formed from stainless steel. The release members 360 of the clipping system 300 generally do not need to bend or straighten during operation, different from the release members 140 of the clipping system 100.

In the retaining position, the release members 360 function to retain the side portions 318 of the jaws 311 within the recess 345 of the distal couplings 340 by blocking the window 346 and preventing the side portions 318 from moving out of the recesses 345. However, the release members 360 allow the side portions 318 to translate and rotate within the recesses 345 as the jaws 311 are opened and closed while resisting the torsional forces imposed during these operations. Accordingly, the jaws 311 can transition from the open configuration (insertion configuration) to the closed configuration (deployment configuration) and from the closed configuration to the open configuration while the release members 360 remain in the retaining position. Any rotational or translational forces of the side portion 318 of the jaw 311 against the release member 360 will not cause the retaining portion (e.g., the third part 368) of the release member 360 to break, withdraw, or otherwise release the clip 310 during one or multiple successive opening and closing operations.

With the retaining force removed, the side portions 318 of the jaws 311 are permitted to exit the recess 345 of the distal coupling 340. With the clip 310 closed over target tissue, the distal couplings 340 can be withdrawn proximally relative to the clip 310 via the extending members 330.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

What is claimed is:

1. A clipping system for treating tissue, comprising:
an adapter including a proximal portion configured to be mounted over a distal end of an insertion device and a distal portion extending distally from the proximal portion;
a clip configured to be mounted over the distal portion of the adapter in an insertion configuration, the clip including first and second jaws connected to one another such that the first and second jaws are movable between the insertion configuration, in which the first and second jaws extend about opposing portions of the distal portion of the adapter and are separated from one another to receive tissue therebetween, and an initial deployment configuration, in which the clip is moved distally off of the adapter so that the first and second jaws are drawn toward one another to grip the tissue therebetween, the first and second jaws being biased toward the initial deployment configuration;
a distal coupling including a channel extending therethrough and a recess formed in a side of the distal coupling, the recess sized and shaped to receive a side portion of the clip therewithin, the recess accessible by a window in the side of the distal coupling; and
a release member sized and shaped to extend at least partially through the channel of the distal coupling, the release member including a retaining portion that, in a retaining position, is positioned within the recess between the window and the side portion of the clip, the retaining portion configured to retain the side portion of the clip within the recess by blocking the side portion of the clip from exiting the recess through the window, the release member movable to a release position in which the retaining portion is withdrawn from the recess to permit the side portion of the clip to exit the recess through the window to decouple the clip from the clipping system.

2. The clipping system of claim 1, wherein the clip includes an opening through which, in the retaining position, the retaining portion of the release member extends, wherein, in the initial deployment configuration, a distal portion of the distal coupling is distal to a first surface of the clip, a proximal portion of the distal coupling is proximal to a second surface of the clip, and the side portion of the clip is received within the recess of the distal coupling.

3. The clipping system of claim 2, further comprising:
an extending member fixed at a distal end thereof to a proximal end of the distal coupling,
wherein decoupling the clip from the clipping system comprises withdrawing the extending members proximally so that the distal coupling is withdrawn proximally relative to the clip while the clip remains closed around target tissue.

4. The clipping system of claim 3, wherein the extending members and the release members are movable together proximally or distally by a single actuator when the clip is in the insertion configuration, the initial deployment configuration, and a review configuration, and
wherein the release members are actuated independently from the extending members to transition to the release position.

5. The clipping system of claim 4, wherein the distal coupling comprises an angled surface on a distal side of the recess that urges the distal coupling to rotate against the side portion of the clip as the distal coupling is withdrawn proximally relative to the clip, the rotation permitting further proximal motion of the distal coupling until the side portion passes outside the recess and the clip is released from the distal coupling.

6. The clipping system of any one of claim 1-claim 5, wherein a distal portion of the release member including the retaining portion is unanchored, wherein lateral forces against the release member by the side portion of each of the first and second jaws are insufficient to overcome frictional forces between the release member, the side portion of each of the first and second jaws and the distal coupling, wherein the release member includes a lateral stiffness sufficient to withstand torsional forces imposed by the side portion of the clip as the side portion of the clip rotates or translates during transitions between the insertion configuration and the initial deployment configuration.

7. The clipping system of claim 6, wherein the distal portion of the release member includes a hook portion sized and shaped to wrap around the side portion of the clip, the hook portion including a first part on a first side of the side portion, a second part curving around a second side of the side portion, and a third part comprising the retaining portion on the second side of the side portion.

8. The clipping system of claim 7, wherein the first side of the side portion of the clip includes a first groove for guiding the first part of the hook portion of the release member and a third side of the side portion of the clip includes a second groove for guiding the third part of the hook portion of the release member.

9. The clipping system of claim 8, wherein moving the release member to the release position comprises advancing the release member distally so that the third part of the release member is withdrawn from the recess.

10. The clipping system of claim 9, wherein the distal coupling includes a cap portion including a slot, wherein advancing the release member distally comprises advancing the hook portion distally into the slot.

11. The clipping system of claim 6, wherein moving the release member to the release position comprises retracting the release member proximally so that the distal portion of the release member is retracted proximal to the recess.

12. The clipping system of any one of claim 1-claim 11, wherein the release member is formed of Nitinol wire.

13. The clipping system of any one of claim 1-claim 12, wherein the release member is formed of suture, wire, or hypotube.

14. The clipping system of any one of claim 1-claim 13, wherein the release member includes the retaining portion formed of a first material and a proximal portion formed of a second material.

15. The clipping system of any one of claim 1-claim 14, wherein the clip can be moved into a review configuration in which the clip is moved distally relative to the adapter.

16. A clipping system for treating tissue, comprising:
an adapter including a proximal portion configured to be mounted over a distal end of an insertion device and a distal portion extending distally from the proximal portion;
a clip configured to be mounted over the distal portion of the adapter in an insertion configuration, the clip including first and second jaws connected to one another such that the first and second jaws are movable between the insertion configuration, in which the first and second jaws extend about opposing portions of the distal portion of the adapter and are separated from one another to receive tissue therebetween, and an initial deployment configuration, in which the clip is moved distally off of the adapter so that the first and second jaws are drawn toward one another to grip the tissue therebetween, the first and second jaws being biased toward the initial deployment configuration;
a distal coupling including a channel extending therethrough and a recess formed in a side of the distal coupling, the recess sized and shaped to receive a side portion of the clip therewithin, the recess accessible by a window in the side of the distal coupling; and
a release member sized and shaped to extend at least partially through the channel of the distal coupling, the release member including a retaining portion that, in a retaining position, is positioned within the recess between the window and the side portion of the clip, the retaining portion configured to retain the side portion of the clip within the recess by blocking the side portion of the clip from exiting the recess through the window, the release member movable to a release position in which the retaining portion is withdrawn from the recess to permit the side portion of the clip to exit the recess through the window to decouple the clip from the clipping system.

17. The clipping system of claim 16, wherein the clip includes an opening through which, in the retaining position, the retaining portion of the release member extends, wherein, in the initial deployment configuration, a distal portion of the distal coupling is distal to a first surface of the clip, a proximal portion of the distal coupling is proximal to a second surface of the clip, and the side portion of the clip is received within the recess of the distal coupling.

18. The clipping system of claim 17, further comprising:
an extending member fixed at a distal end thereof to a proximal end of the distal coupling,
wherein decoupling the clip from the clipping system comprises withdrawing the extending members proximally so that the distal coupling is withdrawn proximally relative to the clip while the clip remains closed around target tissue.

19. The clipping system of claim 18, wherein the extending members and the release members are movable together proximally or distally by a single actuator when the clip is in the insertion configuration, the initial deployment configuration, and a review configuration, and
wherein the release members are actuated independently from the extending members to transition to the release position.

20. The clipping system of claim 19, wherein the distal coupling comprises an angled surface on a distal side of the recess that urges the distal coupling to rotate against the side portion of the clip as the distal coupling is withdrawn proximally relative to the clip, the rotation permitting further proximal motion of the distal coupling until the side portion passes outside the recess and the clip is released from the distal coupling.

21. The clipping system of claim 16, wherein a distal portion of the release member including the retaining portion is unanchored, wherein lateral forces against the release member by the side portion of each of the first and second jaws are insufficient to overcome frictional forces between the release member, the side portion of each of the first and second jaws and the distal coupling, wherein the release member includes a lateral stiffness sufficient to withstand torsional forces imposed by the side portion of the clip as the side portion of the clip rotates or translates during transitions between the insertion configuration and the initial deployment configuration.

22. The clipping system of claim 21, wherein the distal portion of the release member includes a hook portion sized and shaped to wrap around the side portion of the clip, the hook portion including a first part on a first side of the side portion, a second part curving around a second side of the side portion, and a third part comprising the retaining portion on the second side of the side portion.

23. The clipping system of claim 22, wherein the first side of the side portion of the clip includes a first groove for guiding the first part of the hook portion of the release member and a third side of the side portion of the clip includes a second groove for guiding the third part of the hook portion of the release member.

24. The clipping system of claim 23, wherein moving the release member to the release position comprises advancing the release member distally so that the third part of the release member is withdrawn from the recess.

25. The clipping system of claim 24, wherein the distal coupling includes a cap portion including a slot, wherein advancing the release member distally comprises advancing the hook portion distally into the slot.

26. The clipping system of claim 21, wherein moving the release member to the release position comprises retracting the release member proximally so that the distal portion of the release member is retracted proximal to the recess.

27. The clipping system of claim 16, wherein the release member is formed of Nitinol wire.

28. The clipping system of claim 16, wherein the release member is formed of suture, wire, or hypotube.

29. The clipping system of claim 16, wherein the release member includes the retaining portion formed of a first material and a proximal portion formed of a second material.

30. The clipping system of claim 16, wherein the clip can be moved into a review configuration in which the clip is moved distally relative to the adapter.

31. A method for treating tissue, comprising:
inserting to a target area in a body lumen, a clip mounted over a distal portion of an adapter, the adapter including a proximal portion configured to be mounted over a distal end of an insertion device and the distal portion extending distally from the proximal portion, the clip being maintained in an insertion configuration in which first and second jaws extend about opposing portions of the distal portion of the adapter and are separated from one another to receive tissue therebetween, the clip being coupled to the adapter by a distal coupling including a channel extending therethrough and a recess formed in a side of the distal coupling, the recess sized and shaped to receive a side portion of the clip therewithin, the recess accessible by a window in the side of the distal coupling, and a release member sized and shaped to extend at least partially through the channel of the distal coupling, the release member including a retaining portion that, in a retaining position, is positioned within the recess between the window and the side portion of the clip, the retaining portion configured to retain the side portion of the clip within the recess by blocking the side portion of the clip from exiting the recess through the window;
drawing the tissue into a channel of the adapter and between the first and second jaws of the clip;
moving the clip distally off of the adapter to permit the clip to move from the insertion configuration to an initial deployment configuration, the first and second jaws of the clip being biased toward the initial deployment configuration in which the first and second jaws extend toward one another to grip the tissue drawn into the adapter;
moving the release member to a release position in which the retaining portion is withdrawn from the recess to permit the side portion of the clip to exit the recess through the window; and
drawing the distal coupling proximally to decouple the clip from a clipping system.

32. The method of claim 31, wherein the clip includes an opening through which, in the retaining position, the retaining portion of the release member extends, wherein, in the initial deployment configuration, a distal portion of the distal coupling is distal to a first surface of the clip, a proximal portion of the distal coupling is proximal to a second surface of the clip, and the side portion of the clip is received within the recess of the distal coupling.

33. The method of claim 31, wherein a distal portion of the release member including the retaining portion is unanchored, wherein lateral forces against the release member by the side portion of each of the first and second jaws are insufficient to overcome frictional forces between the release member, the side portion of each of the first and second jaws and the distal coupling, wherein the release member includes a lateral stiffness sufficient to withstand torsional forces imposed by the side portion of the clip as the side portion of the clip rotates or translates during transitions between the insertion configuration and the initial deployment configuration.

34. The method of claim 33, wherein the distal portion of the release member includes a hook portion sized and shaped to wrap around the side portion of the clip, the hook portion including a first part on a first side of the side portion, a second part curving around a second side of the side portion, and a third part comprising the retaining portion on the second side of the side portion, wherein moving the release member to the release position comprises advancing the release member distally so that the third part of the release member is withdrawn from the recess.

35. The method of claim 33, wherein moving the release member to the release position comprises retracting the release member proximally so that the distal portion of the release member is retracted proximal to the recess.
